(19) United States
(12) Patent Application Publication
(10) Pub. No.: US 2015/0071412 A1
(43) Pub. Date: Mar. 12, 2015

(54) C-ARM RETENTION LOCKS, SYSTEMS CONTAINING THE SAME, AND METHODS FOR USING THE SAME

(71) Applicant: General Electric Company, Schenectady, NY (US)

(72) Inventors: John Matthew Simmons, Salt Lake City, UT (US); David Ellis Barker, Salt Lake City, UT (US); Krishnanand Prabhu Bailur, Bangalore (IN); Allen Michael Collins, Salt Lake City, UT (US)

(73) Assignee: General Electric Company, Schenectady, NY (US)

(21) Appl. No.: 14/020,600

(22) Filed: Sep. 6, 2013

(51) Int. Cl. A61B 6/10; A61B 6/00

(52) U.S. Cl. CPC A61B 6/105; A61B 6/4441
USPC 378/198; 403/325

(57) ABSTRACT

C-arm retention locks allowing users to secure and unsecure C-arm positioning devices of an X-ray machine, systems containing such locks, and methods of using such locks are described. The C-arm retention lock comprises a latch bail and a latching assembly. The latching assembly comprises a latch base having an ear and a generally flat engagement surface at an end of the latch base proximate the ear, a latch having an arm, the latch being pivotally connected to the latch base so the latch is rotatable between closed and open positions, and a tensioning device engaging the latch and latch base such that the tensioning device pulls the latch toward either of the closed or open positions. The latching assembly or latch bail is configured to be connected to a C-arm and the latching assembly retains the latch bail between the ear and the arm when the latch is closed.

### FIELD

This application relates generally to X-ray equipment. More specifically, this application relates to C-arm retention locks that allow users to easily secure and unsecure a C-arm positioning device of an X-ray machine, systems containing such locks, and methods of using such locks.

### BACKGROUND

A typical X-ray imaging system comprises an X-ray source and an X-ray detector. X-rays emitted from the X-ray source can impinge on the X-ray detector and provide an X-ray image of an object or subject that is placed between the X-ray source and the detector. In one type of X-ray imaging system, a fluoroscopic imaging system, the X-ray detector is often an image intensifier or a flat panel digital detector.

Fluoroscopic imaging systems can be either fixed or mobile. For instance, fixed fluoroscopic imaging systems often include a gantry that is secured to a floor, wall, or ceiling. Additionally, mobile fluoroscopic imaging systems are movable so that they can be used in a variety of clinical environments, such as radiology and surgery departments of a medical facility. The mobile fluoroscopic imaging systems may include a C-arm, G-arm, O-arm, L-arm, or another imaging arm or gantry assembly. A mobile fluoroscopic imaging system may also be configured as a mini C-arm.

In some configurations, a C-arm assembly of a fluoroscopic imaging system moves relative to the subject in order to acquire images from multiple angles. In some arrangements, the C-arm assembly is manually repositioned to generate images from different angles. In some configurations, the C-arms are connected to one end of a movable arm so the C-arm can often be raised and lowered, be moved from side to side, and/or be rotated about one or more axes of rotation. Accordingly, such C-arms can be moved and reoriented to allow X-ray images to be taken from several different positions and angles and of one or more portions of a patient, without requiring the patient to be frequently repositioned. Retention systems are often used to restrain C-arms when the X-ray machine is not in use or during transport of the X-ray machine.

### SUMMARY

This application generally describes C-arm retention locks that allow users to easily secure and unsecure a C-arm positioning device of an X-ray machine, systems containing such locks, and methods of using such locks. This application specifically describes a C-arm retention lock, comprising a latch bail and a latching assembly, wherein the latching assembly comprises: a latch base having an ear and a generally flat engagement surface at an end of the latch base proximate the one ear; a latch having an arm, the latch being pivotally connected to the latch base so that the latch is rotatable between a closed and open position; and a tensioning device engaging the latch and the latch base such that the tensioning device pulls the latch toward either of the closed or open positions, where the latching assembly or the latch bail is configured to be connected to a C-arm, and the latching assembly retains the latch bail between the ear and the arm when the latch is in the closed position. Thus, the C-arm retention lock can allow a user to easily secure and release the C-arm to a mobile or fixed X-ray machine.

### BRIEF DESCRIPTION OF THE DRAWINGS

The following description can be better understood in light of the Figures, in which:

FIGS. 1A and 1B show a front perspective view of some embodiments of a C-arm retention lock device;

FIG. 2 shows an exploded side perspective view of some embodiments of a latching assembly of the C-arm retention lock device;

FIG. 3 shows a bottom view of some embodiments of a latching assembly;

FIG. 4 shows a perspective view of some embodiments of a latch;

FIG. 5 shows a perspective view of some embodiments of a tensioning device retainer;

FIG. 6 shows some embodiments of a latch bail;

FIG. 7 shows a perspective view of some embodiments of a latching assembly;

FIG. 8 shows a cross-sectional side view of some embodiments of a latch bail and a latching assembly in an open position;

FIG. 9 shows a cross-sectional side view of some embodiments of a latch bail and A latching assembly in a closed position;

FIGS. 10-11 show some embodiments of a different configuration of a latching assembly and latch.

The Figures illustrate specific aspects of the described C-arm retention lock devices and methods for making and using such devices. Together with the following description, the Figures demonstrate and explain the principles of the methods and structures produced through these methods. In the drawings, the thickness of layers and regions are exaggerated for clarity. The same reference numerals in different drawings represent the same element, and thus their descriptions will not be repeated. As the terms on, attached to, or coupled to are used herein, one object (e.g., a material, a layer, a substrate, etc.) can be on, attached to, or coupled to another object regardless of whether the one object is directly on, attached, or coupled to the other object or there are one or more intervening objects between the one object and the other object. Also, directions (e.g., above, below, top, bottom, side, up, down, under, over, upper, lower, horizontal, vertical, “x,” “y,” “z,” etc.), if provided, are relative and provided solely by way of example and for ease of illustration and discussion and not by way of limitation. In addition, where reference is made to a list of elements (e.g., elements a, b, c), such reference is intended to include any one of the listed elements by itself, any combination of less than all of the listed elements, and/or a combination of all of the listed elements.

### DETAILED DESCRIPTION

The following description supplies specific details in order to provide a thorough understanding. Nevertheless, the skilled artisan will understand that the described C-arm retention lock devices and associated methods of making and using the devices can be implemented and used without employing these specific details. Indeed, the C-arm retention lock devices and associated methods can be placed into practice by modifying the described devices and methods and can be used in conjunction with any other apparatus and techniques conventionally used in the industry. For example, while the description below focuses on methods for making and using the C-arm retention lock devices for mini C-arms, they can be used with virtually any other type of X-ray equipment, including C-arms, G-arms, O-arms, and L-arms.

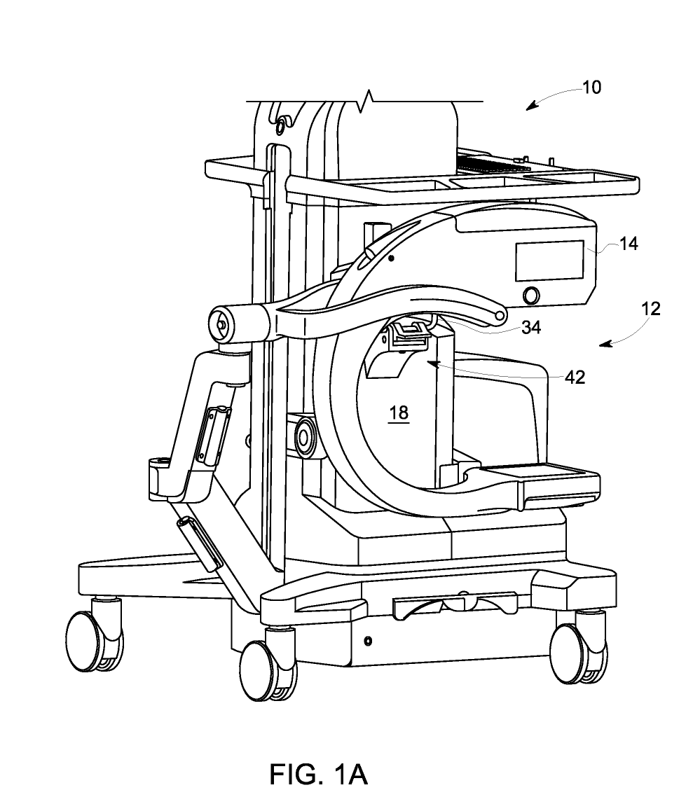

The C-arm retention locks (or just locks) can comprise any suitable component to easily secure and unsecure a C-arm positioning device of an X-ray machine. FIGS. 1A and 1B show some embodiments in which the C-arm lock 12 can be mounted on an X-ray machine 10. In the illustrated embodiments, the C-arm positioning device 14 may be a sliding counterbalanced C-arm positioning device that is part of the mobile x-ray machine 10.

The C-arm lock 12 comprises a latch bail 34 and a docking (or latching) assembly 42 that are used for the C-arm positioning device (or C-arm) 14. In some embodiments, the latch bail 34 can mounted to the C-arm and the docking or latching assembly 42 can be mounted to a housing 18 of the X-ray machine. In other embodiments, the latch bail 34 can be mounted to the housing body 18 and the docking or latching assembly 42 can be mounted to the C-arm 14.

Some embodiments of the C-arm 14, latch bail 34, and latching assembly 42 are illustrated in FIG. 1B. In these embodiments, the latch bail 34 can be mounted to and extend downwards from an inner surface of the C-arm 14 and oriented to be substantially parallel to a surface of the housing 18 of the x-ray machine 10. The latch bail 34 contains a rod 38 that is oriented generally horizontal and parallel with the side of the housing 18.

As show in FIG. 1B, the latching assembly 42 can comprise a latch base 46 connected to a latch 50. The latch base 46 may be connected by any number of different methods to the housing 18 or may be integral to the housing 18. In some configurations, the latch base 46 may be connected to any other grounded or secured mounting surface proximate to the C-arm 14. Since the latch bail 34 can be received within the latching assembly 42 between the latch base 46 and the latch 50 such that the movement of the C-arm 14 is limited in any direction, the latching assembly 42 may be opened such that the latch bail 34 is released and the C-arm 14 may be moved.

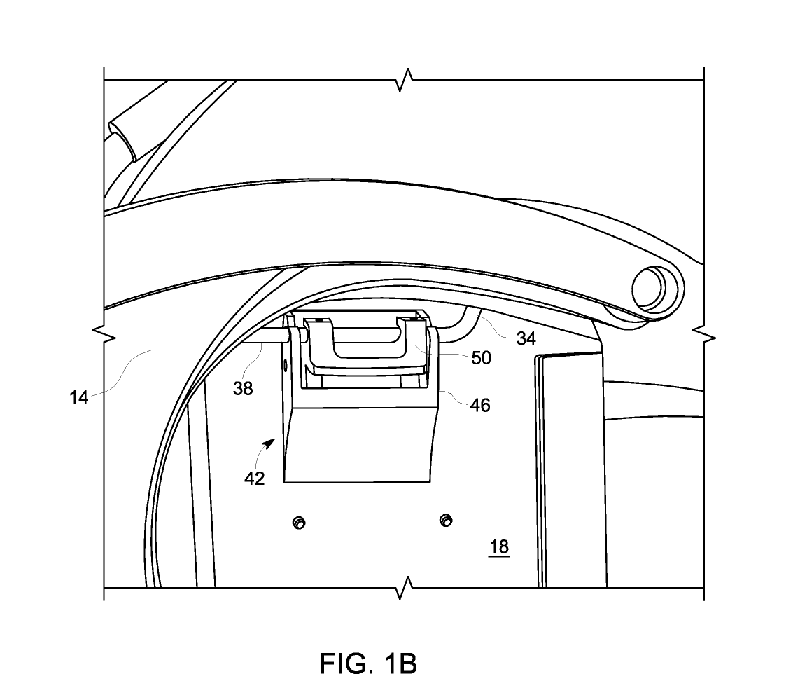

FIGS. 2 to 5 show some embodiments of components and arrangements of the latching assembly 42. In these embodiments, the latching assembly 42 comprises three components: a latch base 46; a latch 50; and a tensioning device assembly 132. These components of the latching assembly 42 may comprise plastic or metal or any number or combination of materials. By way of example, the latching assembly 42 can be made from any substantially rigid material, including aluminum or plastic.

FIG. 2 illustrates some embodiments of components and arrangements of the first component of the latching assembly 42, the latch base 46. In these embodiments, the latch base 46 comprises a base plate 54 extending from one end and a latching section 62 extending from the opposite end. The latching section 62 includes pairs of rounded ears 70 extending out at opposite sides of the latching section 62. A cutout 72 defined by a stop surface 78 that extends between the ears 70 along the latching section 62. The latching section 62 can contain a limiting surface 74 that extends downward and perpendicular from the stop surface 78 away from the ears 70. The stop surface 78 includes a spring aperture 82 extending therethrough. The stop surface 78 keeps the latch 50 from continuing to rotate. The ears 70 include pinholes 96 extending therethrough to the cutout 72. The ears 70 can have a wear-impact surface 86 extending along the surfaces thereof. The base plate 54 further includes apertures 66 through which screws or other fasteners may be inserted to connect the latch base 46 to the housing body 18. The latch base 46 includes a stop surface 90 proximate the latching section 62. During operation, the latching assembly 42 receives the latch bail 34 of the C-arm 14 (as shown in FIG. 1) along the latching section 62 between the pairs of rounded ears 70. The wear impact surfaces 86 on the ears 70 operate to protect both the latch base 46 and handle shaped latch bail 34 from wear.

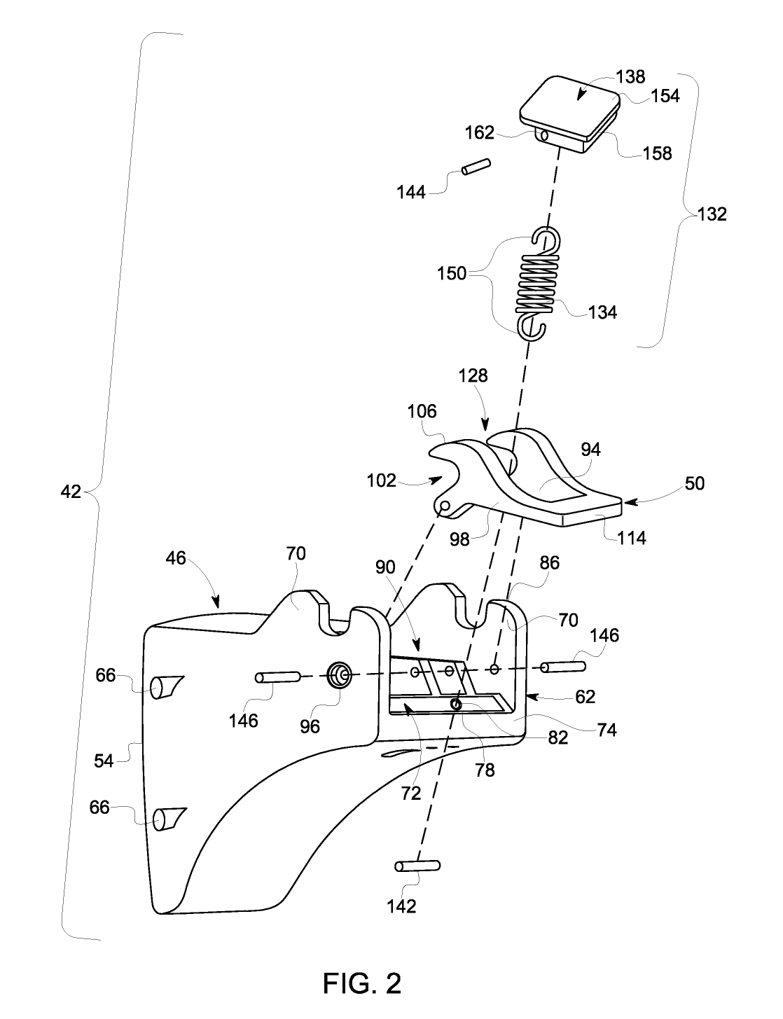

FIG. 3 illustrates a bottom view of the latch base 46. On one end of the latch base 46, the base plate 54 abuts the housing body 18 and connects the latch base 46 to the housing body 18. The latching section 62 is located on the opposite end of the latch base 46 from the base plate 54. The underside of the latching section 62 includes a thin gap 186 along a bottom surface 190 thereof. The thin gap 186 perpendicularly traverses the spring aperture 82. The gap 186 is partly defined by ledges 194 on opposite sides thereof such that the gap 186 can receive and retain a pin 142 (FIG. 2) therein against the ledges 194. The pin 142 extends through a hook 150 of a spring 134 that extends through the spring aperture 82.

The second component of the latching assembly 42 is the latch 50. Some embodiments of the latch 50 are shown in FIG. 2. In these embodiments, the latch 50 comprises a base 94 with curved arms 98. The arms 98 project from opposite sides thereof to define gaps 102 between the arms 98 and the base 94. In some configurations, the arms 98 have wear-impact surfaces 106 extending along contact surfaces thereof. The base 94 includes a curved handle 114 extending therefrom proximate to the arms 98.

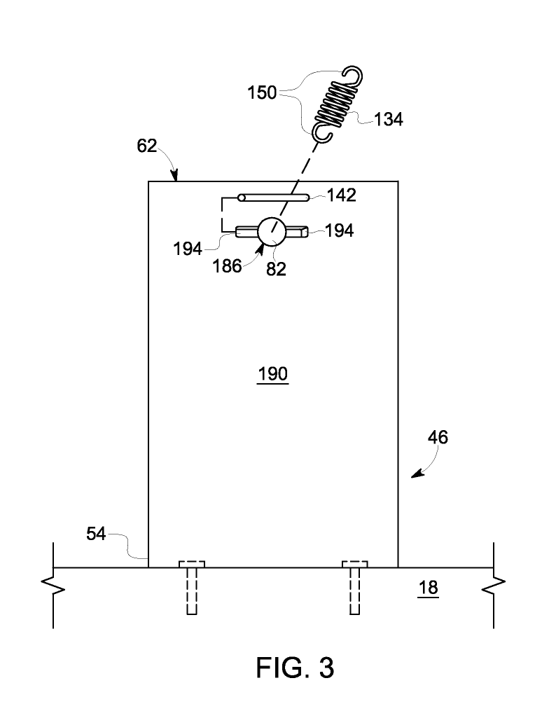

FIG. 4 illustrates further details of the latch 50 of FIG. 2. The base 94 of the latch 50 has a square hole 128. The base 94 also has a bottom side 130 from which extends a rounded pivot section 118. The pivot section 118 includes an aperture 122 extending therethrough. A stop bar 126 also extends from the bottom side 130 of the base 94.

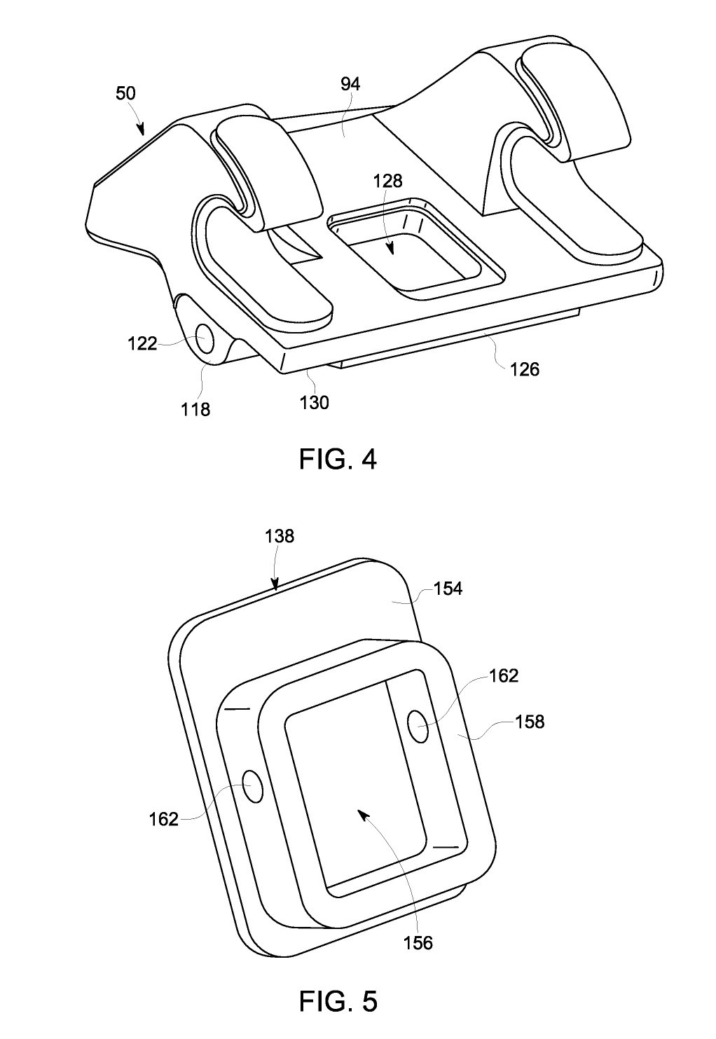

The third component of the latching assembly 42 is the latch tensioning device assembly 132. As shown in FIG. 2, the tensioning device assembly 132 comprises a spring 134 with hooks 150 and a tensioning device retainer 138. One hook 150 of the spring 134 extends through the spring aperture 82 of the latch base 46. The other hook 150 of the spring 134 is retained by the tensioning device retainer 138. FIG. 5 illustrates a bottom isometric view of the tensioning device retainer 138 of FIG. 2. The tensioning device retainer is configured to selectably couple with the square hole 128 of the latch 50. The cap-shaped tensioning device retainer 138 has a base 154 from which extends a square-shaped wall 158 defining a hollow interior section 156. The wall 158 has apertures 162 extending through parallel sections thereof. The apertures 162 are configured to receive a pin 144 (FIG. 2) to retain one hook 150 of the spring 134. By way of example only, the tensioning device retainer 138 can be made of aluminum.

These three main components of the latching assembly 42 can arranged and assembled as shown in FIG. 2. The latch 50 is selectably coupled to the latch base 46 such that the aperture 122 of the latch 50 aligns with the pin holes 96 of the latch base. Pins 146 are inserted into pin holes 96 and enter aperture 122 to secure the latch 50 to the latch base. Next, a hook 150 of the spring 134 is inserted into the spring aperture 82. Pin 142 is extended through the hook 150 and the pin 142 is received in the gap 186, as shown in FIG. 3. Then, the other hook 150 of the spring 134 is inserted into the interior section 156, as shown in FIG. 5, of the tensioning device retainer 138. The pin 144 is then inserted through the apertures 162 in the tensioning device retainer 138 and the hook 150 to hold the spring 134 to the tensioning device retainer 138. Finally, the tensioning device retainer 138 is then positioned in latch 50 such that the wall 158 is received into the square hole 128 of the latch 50 and the base 154 of the tensioning device retainer 138 engages the base 94 of the latch 50. The spring 134 is in tension between the latch base 46 and the tensioning device retainer 138 and maintains tension between the latch 50 and the latch base 46.

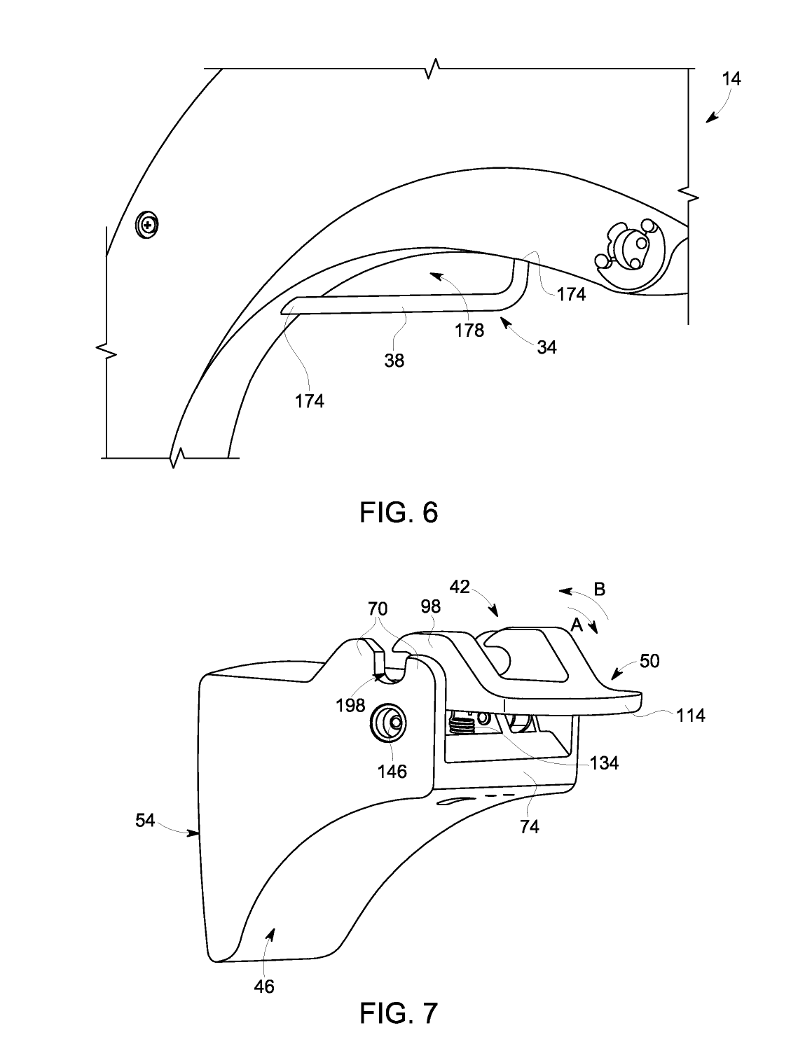

As noted above, the latching assembly 42 works with the latch bail 34 with to secure (and release) the C-arm 14. FIG. 6 illustrates an isometric view of some embodiments of the latch bail 34. In these embodiments, the latch bail 34 is mounted to the C-arm 14 at mounting points 174. The handle-shaped rod 38 extends from an inside surface of the C-arm 14 to define a gap 178. In other embodiments, the latch bail 34 may be connected to the C-arm 14 by any number of methods or may be integral with the C-arm 14. The latch bail 34 may comprise plastic, metal, or composite materials such as carbon fiber or fiberglass. By way of example only, the latch bail 34 may be made of aluminum or stainless steel. The skilled artisan will understand that the latch bail 34 may have any number of different shapes and orientations, for example, the rod 38 of the latch bail 34 may extend horizontally sideways towards the housing body 18.

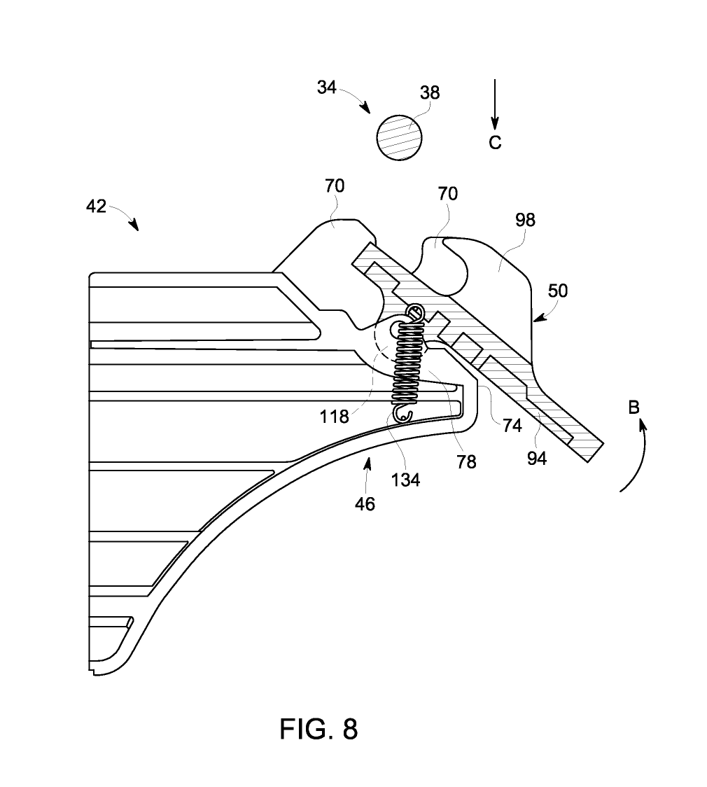

Returning to the latching assembly 42, FIG. 7 illustrates an isometric view of some embodiments of the assembled latching assembly 42 from FIGS. 1-5. In these embodiments, the latching assembly 42 can be assembled such that the latch 50 is in the closed position where the stop bar 126 of the latch 50 engages the stop surface 90 on the latch base 46 and the arms 98 and ears 70 define an enclosed region 198. The spring 134 holds the latch 50 in the closed position. In operation, the latch 50 can rotate about the pins 146 in the direction of arrow A to an open position where the limiting surface 74 of the latch base 46 resistibly engages the handle 114 of the latch 50. When the latch 50 is in the open position, it can be rotated about the pins 146 in the direction of arrow B from the open position back to the closed position. When the latch 50 has been rotated a certain distance from the closed position toward the open position, the spring 134 automatically shifts from pulling the latch 50 toward the closed position and applies a tension to the latch 50 to pull the latch 50 toward the open position. The spring 134 holds the latch 50 in the open position, but as the latch 50 is rotated a certain distance from the open position toward the closed position, the spring 134 automatically shifts from pulling the latch 50 toward the open position and applies a tension to the latch 50 to pull the latch 50 toward the closed position. In this operation, the spring 134 serves as a toggle device that can pull and hold the latch 50 in either position.

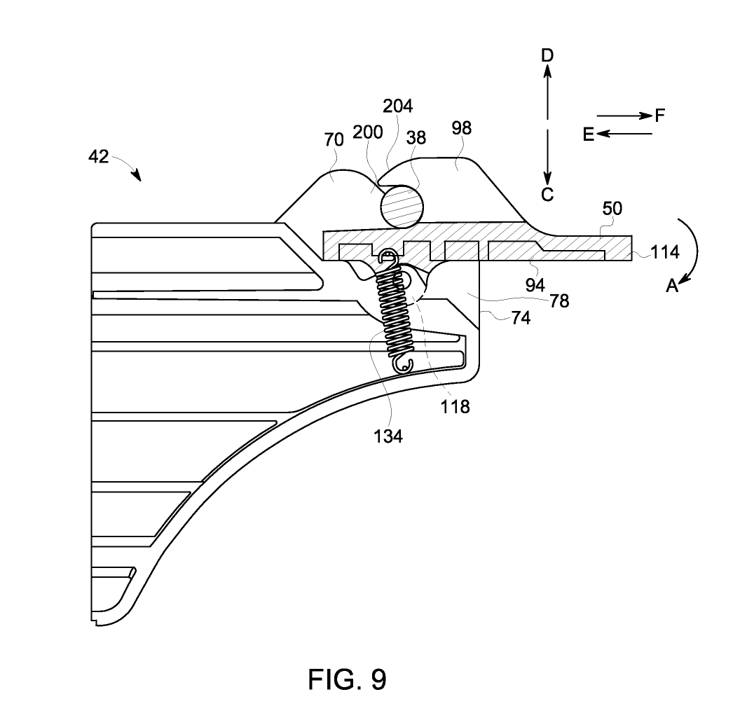

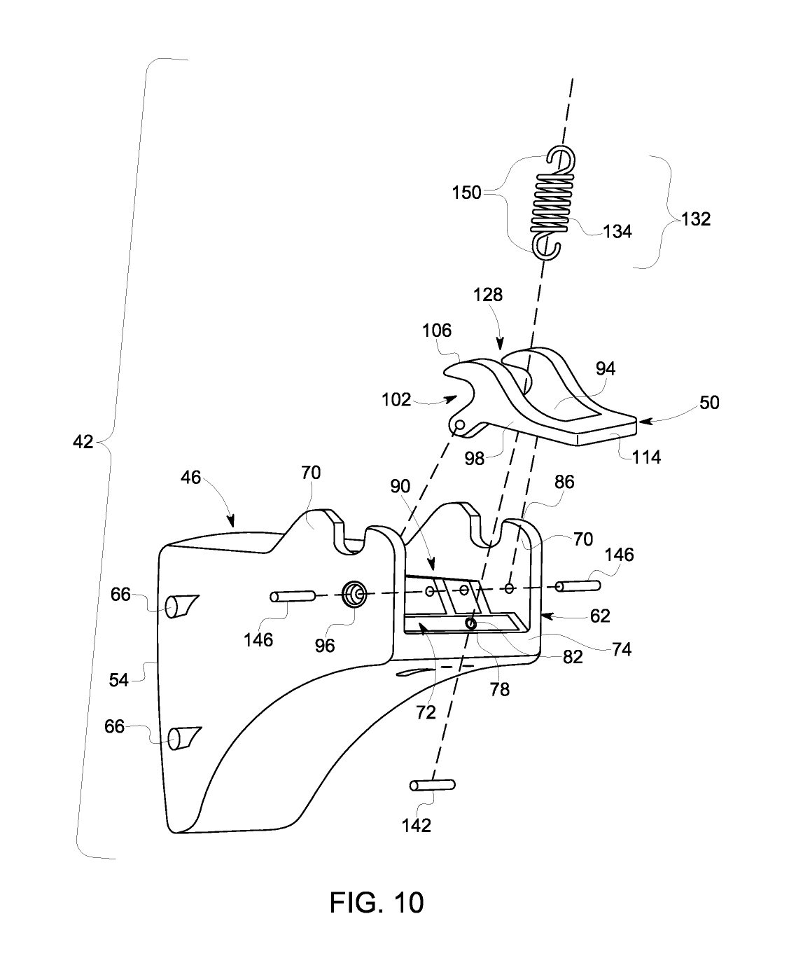

FIGS. 8 and 9 show some embodiments of the open and closed positions of the latching assembly 42 to retain and release the latch bail 34. FIG. 8 illustrates a cross-sectional side view of the latch bail 34 and the latching assembly 42 in the open position. When the latching assembly 42 is in the open position, the base 94 of the latch 50 engages the limiting surface 74 of the latch base 46 and the pivot section 118 of the latch 50 engages the engagement surface 78 of the latch base 46. The base 94 of the latch 50 extends out beyond the ears 70 of the latch base 46 between the ears 70 and the arms 98 of the latch 50. The orientation of pivot section 118 on the engagement surface 78 with respect to the orientation of the spring 134 allows for the spring 134 to be in tension to hold the latch 50 in the open position.

In operation, to dock the latch bail 34 within the latching assembly 42, the C-arm 14 can be moved towards the latching assembly 42 such that the rod 38 of the latch bail 34 is received into the gap between the ears 70 and the rod 38 engages the base 94 of the latch 50. Next, as the rod 38 pushes against the base 94 in the direction of arrow C, the latch 50 is pushed to rotate in the direction of arrow B against the tension of the spring 134. After the latch 50 has rotated a certain distance in the direction of arrow B, the orientation of the pivot section 118 on the engagement surface 78 with respect to the orientation of the spring 134 allows for the spring 134 in tension to pull the latch 50 toward the closed position. The latch 50 then moves to the closed position where the stop bar 126 engages the stop surface 90 of the latch base 46, the gaps 102 of the latch 50 engage the rod 38, and the arms 98 of the latch 50 are received into the gap 178. Therefore, in this operation, the spring 134 serves as a toggle device that can shift from automatically pulling the latch 50 to the open position to pulling the latch 50 to the closed position after the latch 50 has been rotated a certain distance to the closed position.

FIG. 9 illustrates a cross-sectional side view of the latching assembly 42 receiving the latch bail 34 in the closed position. The latch 50 is in the closed position and the rod 38 of the latch bail 34 is received within the enclosed region 198 between the ears 70 of the latch base 46 and the arms 98 of the latch 50. The spring 134 is in tension to hold the latch 50 in the closed position about the latch bail 34. In this manner, the latching assembly 42 generally retains or restrains the C-arm 14 from moving. Specifically, the ears 70 prevent the latch bail 34 from moving in the direction of arrow E or the direction of arrow F by engaging the rod 38. Also, the latch 50 prevents the latch bail 34 from moving in the direction of arrow D because as the rod 38 moves in the direction of arrow D, the rod 38 engages the arm 98 at a pivot point such that the arm 98 does not rotate.

To release the latch bail 34 from the latching assembly 42 so that the C-arm 14 can move, the operator simply depresses the handle 114 of the latch 50 in the direction of arrow A. After the latch 50 has been rotated a certain distance in the direction of arrow A, the orientation of the pivot section 118 on the engagement surface 78 with respect to the orientation of the spring 134 allows for the spring 134 to be in tension to pull the latch 50 toward the open position. The latch 50 then rotates to the open position where the base 94 of the latch 50 engages the limiting surface 74 of the latch base 46. The tension load on the latch 50 as it is rotated toward the open position assists to eject the latch bail 34 during undocking. The operator may then pull the latch bail 34 away from the latching assembly 42.

Alternatively, an operator can secure the latch bail 34 into the latching assembly 42 when the latching assembly 42 in already in the closed position. When the latching assembly 42 is in the closed position as shown in FIG. 9, the C-arm 14 is moved toward the latching assembly 42 such that the rod 38 engages the curved ears 70 at an inside surface or ramp 200 and the curved arms 98 at a top surface or ramp 204 in the general direction of Arrow C. Because the wear impact surfaces 86 and 106 of the ears 70 and arms 98, respectively, are smooth and create a minimal amount of friction, the arms 98 and the ears 70 are able to slidingly guide the rod 38 of the latch bail 34 therebetween such that the latch bail 34 pushes the arms 98, and thus the entire latch 50, in the direction of arrow A toward the open position. After the rod 38 slides past the top surface 204 of the arms 98 and against the base 94 of the latch 50 between the arms 98 and the ears 70, the latch 50 is pulled back into the closed position by the spring 134 to secure the latch bail 34 in the latching assembly 42. Thus, the operator can secure the latch bail 34 even when the latching assembly 42 is already in the closed position. And the operator need not even touch the latch 50 to secure the latch bail 34 into the latching assembly 42.

In alternate embodiments, the latch bail 34 may have any number of other shapes and/or orientations that allow for it to be restrained between the arms 98 and ears 70 of the latching assembly 42. And the latching assembly 42 may be structured and oriented such that it can receive the latch bail 34 from any number of different directions and orientations besides from above in the downward direction of Arrow C. For example, the latching base 46 and the latch 50 may be structured to extend outward in the direction opposite of the C-arm 14 and receive the latch bail 34 from the direction of Arrow E.

In other embodiments, the ears 70 of the latch base 46 and the arms 98 of the latch 50 may overlap each other or contact each other or one may be received within the other when the latch 50 in the closed position. Alternatively, the latch base 46 may include only one ear 70 or more than two ears 70 and the latch 50 may include only one arm 98 or more than two arms 98. Alternatively, the latch base 46 may include the curved arms 98 and the latch 50 may include the rounded ears 70. In yet other embodiments, the latching assembly 42 may be connected to the C-arm 14 and the latch bail 34 may be connected to a mounting surface such that the latching assembly 42 on the C-arm 14 is operated to secure the latching assembly 42 to the latch bail 34 connected to the mounting surface.

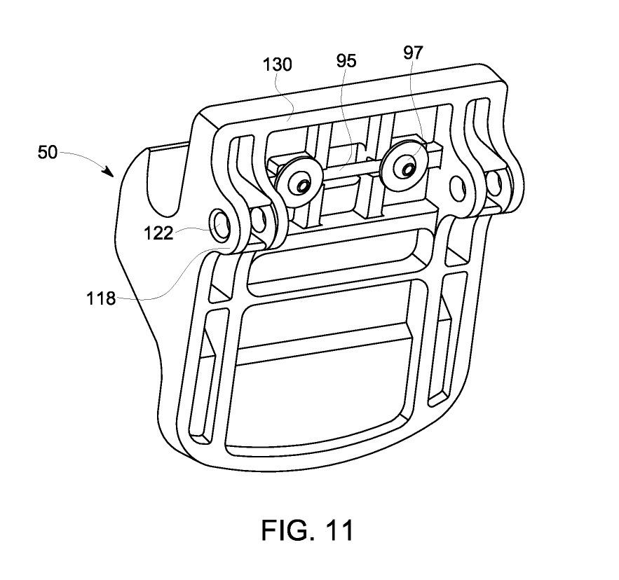

In yet other embodiments, the tensioning device assembly 132 and latch 50 can be configured differently as illustrated in FIGS. 10-11. In these embodiments, the tensioning device retainer 138 and the pin 144 have been eliminated as shown in FIG. 10. The spring 134 continues to be used in these configurations. As shown in FIG. 11, the latch 50 can be modified so that instead of tensioning device retainer 138, the bottom of the base 94 of the latch 50 contains a pin 95 on which the hook 150 is retained. The pin 95 is connected to the base 94 using anchoring elements 97 located on the each end of the pin 95. Thus, rather than being connected through the latch 50, the spring 134 is connected on the bottom of the latch 50.

The C-arm lock 12 can comprise any other component or characteristic that allows it to be secured and released. For example, the C-arm lock 12 can comprise one or more support structure covers, handles, power supplies (e.g., internal and/or external), control devices, actuators, and/or brake systems.

The described C-arm lock 12 can be made in any manner that forms the structures described. In some embodiments, a latch bail 34 and a latching assembly 42 can be manufactured with the structures described herein and then one of them is attached to the C-arm 14 and the other attached to the x-ray machine.

The C-arm lock 12 can also be used to then secure or release the C-arm 14 to the x-ray machine 10. By way of example, an operator can secure the C-arm lock 12 by moving the C-arm 14 such that the latch bail 34 is aligned over the latching assembly 42 and applying downward force to engage the C-arm lock 12. With the C-arm lock 12 engaged, the operator can transport or store the X-ray machine 10 with the C-arm 14 secured. Likewise, the operator can unsecure the C-arm lock 12 by applying downward force on the handle 114 of the latch 50 to disengage the C-arm lock. With the C-arm lock disengaged, the operator can position the C-arm 14 as required. By way of further example, the operator can secure the C-arm lock 12 with the latch 50 already in the closed position by aligning the latch bail 34 over the latching assembly 42 and applying downward force to move the latch 50 into the open position to accept the latch bail thereby engaging the C-arm 14 and subsequently moving the latch 50 into the closed position.

The C-arm lock 12 may have several useful features. First, because the latch bail 34 is restrained from moving in the direction of arrows C, D, E, and F, the C-arm lock 12 adequately secures C-arm 14 for transport or storage of X-ray machine 10. Accordingly, the C-arm lock 12 sufficiently immobilizes C-arm 14 to protect the C-arm 14 and the X-ray machine 10 during transport or storage of the X-ray machine 10. The C-arm lock 12 also secures the C-arm 14 during transport or storage to prevent injury to the operator or patients. The C-arm lock 12 also allows for the C-arm to be secured to ease transport of the X-ray machine. With the C-arm 14 secured by the C-arm lock 12, an operator can freely move the X-ray machine 10 without the C-arm swinging free and causing damage or injury. Also, the C-arm lock 12 secures the C-arm 14 to allow for a single operator to move the X-ray machine 10 without needing additional operators to secure or guide the C-arm 14.

Second, the C-arm lock 12 is easy and intuitive to use. The operator can operate the C-arm lock 12 to secure and unsecure the C-arm 14 with only a minimum of training. The C-arm lock 12 is intuitive in that the C-arm 14 is easily secured by applying downward pressure to secure the C-arm lock 12 in the motion most intuitive for securing the C-arm 14. Also, to secure the C-arm lock 12, an operator does not even need to touch the latch or latching assembly. To secure the C-arm lock 12, the operator only needs to align the latch bail 34 with the latching assembly 42 and exert a downward pressure to secure the C-arm lock 12. Additionally, in some embodiments, the operator only needs to use one hand to secure and unsecure the C-arm lock 12. In some procedures in which the operator must maintain sterility of at least one of the operator's hands, the operator can secure and unsecure the C-arm lock 12 with one hand while maintaining sterility of the other hand. Further, the C-arm lock 12 can be secured even when the latch 50 is in the closed position, thereby eliminating a separate step to open the latch 50 before securing the C-arm lock 12.

Third, the wear surfaces of the C-arm lock 12 are not painted thereby preventing damage or scratching to the painted or finished surfaces of the X-ray machine 10 when the C-arm lock is secured or unsecured. To prevent damage to the painted or finished surfaces of the X-ray machine, the latch bail 34 and the wear surfaces of the latching assembly 42 are not painted. As these are the only components that come into contact when the C-arm lock 12 is secured or unsecured, damage or scratching of the painted or finished surfaces of the remaining components of the X-ray machine 10 are prevented. Also, since there is no damage or scratching of the painted or finished surfaces of the X-ray machine 10, paint debris or finish debris is eliminated, thereby reducing potential contamination from these sources. Further, in addition to preventing damage to the paint or finish, the C-arm lock 12 lends an aesthetically pleasing look to the X-ray machine 10.

Fourth, the C-arm lock 12 allows for flexibility in placement of latching assembly 42 and the latch bail 34. In some embodiments, the latching assembly 42 can be attached directly to the housing body 18 or to a cover or another component of the X-ray machine 10. Likewise the latch bail 34 can be attached to the C-arm 14 in various configurations. By way of example only, the latch bail 34 can be attached to the inner surface of the C-arm 14 and oriented either horizontally or vertically, or the latch bail 34 can be attached to an outer circumference of the C-arm 14, or the latch bail 34 can be attached to a side of the C-arm 14. In some embodiments, the latch bail 34 can be attached to the X-ray machine 10 and the latching assembly 42 can be attached to the C-arm 14. Furthermore the flexibility in placement of the latching assembly 42 and the latch bail 34, allows for on to be placed on the C-arm 14 and for one to be placed on a structure proximate to the X-ray machine 10. In short, the C-arm lock 12 allows for great flexibility in the placement and arrangement of the latching assembly 42 and the latch bail 34.

Fifth, in some embodiments, the C-arm lock 12 is unobtrusive to the operator. The C-arm lock 12 has no features or protrusions that can catch or snag clothing, exam gloves, linens, electrical cords or cables, sterile drapes, or other materials. This unobtrusiveness prevents injury or inconvenience to the operator, other medical staff and/or patients. This feature also prevents interfering with sterility procedures by reducing or eliminating the possibility of entangling a sterile drape or other sterile measure with any component of the C-arm lock 12. Also, the latch bail 34 is smooth in some embodiments and eliminates the chance of injury to the operator or a patient. Further, since the C-arm lock 12 can be secured with the latch 50 in either the open or closed position, there is no need for the operator to place the operator's hand or other part of the operator's body in the area of the latching assembly 42 or the latch bail 34 as the C-arm lock 12 is secured. Therefore, the possibility of injury to operator is greatly reduced by the unobtrusiveness of the C-arm lock 12.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

We claim:

1. A C-arm retention lock, comprising:
a latch bail; and
a latching assembly, the latching assembly comprising:
a latch base having an ear and a generally flat engagement surface at an end of the latch base proximate the one ear;
a latch having an arm, the latch being pivotally connected to the latch base so that the latch is rotatable between a closed and open position; and
a tensioning device engaging the latch and the latch base such that the tensioning device pulls the latch toward either of the closed or open positions,
wherein the latching assembly or the latch bail is configured to be connected to a C-arm, and the latching assembly retains the latch bail between the ear and the arm when the latch is in the closed position.

2. The C-arm retention lock of claim 1, wherein the latch has a pivot section that engages and moves with respect to the latch base on the engagement surface.

3. The C-arm retention lock of claim 2, wherein the latch rotates about the pivot section between first and second ranges.

4. The C-arm retention lock of claim 3, wherein when the pivot section is rotated within the first range, the pivot section is oriented with respect to the tensioning device so that the tensioning device pulls the latch to the closed position and wherein when the pivot section is rotated within the second range, the pivot section is oriented with respect to the tensioning device such that the tensioning device pulls the latch to the open position.

5. The C-arm retention lock of claim 1, wherein the latch has a base surface and when the latch is in the open position, the latch bail engages the base surface such that the latch rotates toward the closed position and the tensioning device pulls the latch into the closed position.

6. The C-arm retention lock of claim 1, wherein the arm and ear each have surfaces that guide the latch bail when the latch bail engages the latching assembly in the closed position such that the latch bail moves the latch toward the open position so that the latch bail moves between the ear and the arm.

7. The C-arm retention lock of claim 1, wherein the tensioning device pulls the latch toward the closed position when the latch is rotated within a certain range from the closed position, and wherein the tensioning device pulling the latch toward the open position when the latch is rotated beyond the certain range from the closed position.

8. The C-arm retention lock of claim 1, wherein the tensioning device pulls the latch toward the open position when the latch is rotated within a certain range from the open position, and wherein the tensioning device pulls the latch toward the closed position when the latch is rotated beyond the certain range from the open position.

9. The C-arm retention lock of claim 1, wherein the latching assembly comprises a tensioning device retainer connected to a first end of the tensioning device and engaging the latch, the latch base connected to an opposite second end of the tensioning device, the tensioning device being in tension between the tensioning device retainer and the latch base such that the tensioning device pulls the latch toward the closed position or the open position.

10. The C-arm retention lock of claim 1, wherein the other of the latch bail and the latching assembly that is not connected to the C-arm is configured to be connected to a mounting surface so that when the latch bail is received in the closed latch between the ear and the arm, the C-arm is restrained in its movement away from the mounting surface.

11. An X-ray system, comprising:
an X-ray machine containing a movable C-arm;
a latch bail; and
a latching assembly, the latching assembly comprising:
a latch base having an ear and a generally flat engagement surface at an end of the latch base proximate the one ear;
a latch having an arm, the latch being pivotally connected to the latch base so that the latch is rotatable between a closed and open position; and
a tensioning device engaging the latch and the latch base such that the tensioning device pulls the latch toward either of the closed or open positions,
wherein one of the latching assembly or the latch bail is connected to the C-arm and the other is connected to the X-ray machine, and the latching assembly retains the latch bail between the ear and the arm when the latch is in the closed position.

12. The X-ray system of claim 11, wherein the latch has a base surface and when the latch is in the open position, the latch bail engages the base surface such that the latch rotates toward the closed position and the tensioning device pulls the latch into the closed position.

13. The X-ray system of claim 11, wherein the arm and ear each have surfaces that guide the latch bail when the latch bail engages the latching assembly in the closed position such that the latch bail moves the latch toward the open position so that the latch bail moves between the ear and the arm.

14. The X-ray system of claim 11, wherein the tensioning device pulls the latch toward the closed position when the latch is rotated within a certain range from the closed position, and wherein the tensioning device pulling the latch toward the open position when the latch is rotated beyond the certain range from the closed position.

15. The X-ray system of claim 11, wherein the tensioning device pulls the latch toward the open position when the latch is rotated within a certain range from the open position, and wherein the tensioning device pulls the latch toward the closed position when the latch is rotated beyond the certain range from the open position.

16. The X-ray system of claim 11, wherein the latching assembly comprises a tensioning device retainer connected to a first end of the tensioning device and engaging the latch, the latch base connected to an opposite second end of the tensioning device, the tensioning device being in tension between the tensioning device retainer and the latch base such that the tensioning device pulls the latch toward the closed position or the open position.

17. A method of using an X-ray system, comprising:
providing an X-ray machine containing a movable C-arm;
providing a latch bail; and
providing a latching assembly, the latching assembly comprising:
a latch base having an ear and a generally flat engagement surface at an end of the latch base proximate the one ear;
a latch having an arm, the latch being pivotally connected to the latch base so that the latch is rotatable between a closed and open position; and
a tensioning device engaging the latch and the latch base such that the tensioning device pulls the latch toward either of the closed or open positions,
wherein one of the latching assembly or the latch bail is connected to the C-arm and the other is connected to the X-ray machine, and the latching assembly retains the latch bail between the ear and the arm when the latch is in the closed position.

18. The method of claim 17, wherein the latch has a base surface and when the latch is in the open position, the latch bail engages the base surface such that the latch rotates toward the closed position and the tensioning device pulls the latch into the closed position.

19. The method of claim 17, wherein the arm and ear each have surfaces that guide the latch bail when the latch bail engages the latching assembly in the closed position such that the latch bail moves the latch toward the open position so that the latch bail moves between the ear and the arm.

20. The method of claim 17, further comprising providing a tensioning device retainer connected to a first end of the tensioning device and engaging the latch, the latch base connected to an opposite second end of the tensioning device, the tensioning device being in tension between the tensioning device retainer and the latch base such that the tensioning device pulls the latch toward the closed position or the open position.
